(19) United States
(12) Patent Application Publication
(10) Pub. No.: US 2019/0091474 A1
(43) Pub. Date: Mar. 28, 2019

(54) METHOD AND SYSTEM FOR PREVENTING MOTION SICKNESS

(71) Applicants: BOE Technology Group Co., Ltd., Beijing (CN); Hefei BOE Optoelectronics Technology Co., Ltd., Hefei (CN)

(72) Inventors: Xiaodeng ZENG, Beijing (CN); Xiang WANG, Beijing (CN); Wei XU, Beijing (CN); Jiong HUANG, Beijing (CN); Zhengwei ZHU, Beijing (CN); Quanzhi HAN, Beijing (CN)

(21) Appl. No.: 16/143,759

(22) Filed: Sep. 27, 2018

(30) Foreign Application Priority Data
Sep. 28, 2017 (CN) 201710900173.2

(51) Int. Cl. A61N 1/36; A61M 21/02; A61N 1/04

(52) U.S. Cl. CPC A61N 1/36038; A61M 21/02; A61M 2021/0072; A61N 1/0408; A61N 1/36021

(57) ABSTRACT

The disclosure discloses a method and system for preventing motion sickness, the method includes: obtaining, by a processing circuit, a current motion state of a user; judging, by the processing circuit, whether a start condition of an electromagnetic pulse circuit is met according to the current motion state of the user; and triggering, by the processing circuit, the electromagnetic pulse circuit to generate a micro-current affecting an equalizing pulse of a human semicircular canal via a pulsed electromagnetic field, after determining that the start condition is met.

### CROSS-REFERENCE TO RELATED APPLICATION

This Application claims priority to Chinese Patent Application No. 201710900173.2, filed on Sep. 28, 2017, the content of which is incorporated by reference in the entirety.

### TECHNICAL FIELD

This disclosure relates to the field of electronic technologies and particularly to a method and system for preventing motion sickness.

### DESCRIPTION OF THE RELATED ART

Motion sickness is a general name of carsickness, seasickness, airsickness, and sicknesses caused by motions such as swinging, bumping, rotating and accelerating due to various reasons. Mainly, an organ of equilibration, i.e., vestibular organ, of an inner ear of a human body is excessively sensitive, causing dysfunction of a vegetative nervous system, so that dizziness, headache, nausea, emesis and severely even collapse, shock and other sicknesses occur and a pale face, cold sweat, too quick or too slow heartbeat, reduced blood pressure and the like are accompanied therewith.

### SUMMARY

Embodiments of the disclosure provide a method and system for preventing motion sickness.

In one aspect, the embodiments of the disclosure provide a method for preventing motion sickness, the method includes: obtaining, by a processing circuit, a current motion state of a user; judging, by the processing circuit, whether a start condition of an electromagnetic pulse circuit is met according to the current motion state of the user; and triggering, by the processing circuit, the electromagnetic pulse circuit to generate a micro-current affecting an equalizing pulse of a human semicircular canal via a pulsed electromagnetic field, after determining that the start condition is met.

In some embodiments, the current motion state includes a part or all of: the acceleration, a moving distance in a horizontal direction per unit time, or a moving distance in a vertical direction per unit time.

In some embodiments, judging, by the processing circuit, whether the start condition is met according to the current motion state of the user, includes: if the current motion state includes the acceleration, judging, by the processing circuit, whether a current acceleration of the user is greater than a first acceleration threshold set currently, and if so, determining that the start condition is met, otherwise determining that the start condition is not met; or if the current motion state includes the moving distance in the horizontal direction per unit time, judging, by the processing circuit, whether a current moving distance in the horizontal direction per unit time of the user is greater than a first horizontal distance threshold set currently, and if so, determining that the start condition is met, otherwise determining that the start condition is not met; or if the current motion state includes the moving distance in the vertical direction per unit time, judging, by the processing circuit, whether a current moving distance in the vertical direction per unit time of the user is greater than a first vertical distance threshold set currently, and if so, determining that the start condition is met, otherwise determining that the start condition is not met.

In some embodiments, the processing circuit determines the first acceleration threshold set currently by: taking an acceleration threshold corresponding to a current application scenario as the first acceleration threshold set currently according to a correspondence between application scenarios and acceleration thresholds; or the processing circuit determines the first horizontal distance threshold set currently by: taking a horizontal distance threshold corresponding to a current application scenario as the first horizontal distance threshold set currently according to a correspondence between application scenarios and horizontal distance thresholds; or the processing circuit determines the first vertical distance threshold set currently by: taking a vertical distance threshold corresponding to a current application scenario as the first vertical distance threshold set currently according to a correspondence between application scenarios and vertical distance thresholds.

In some embodiments, after triggering, by the processing circuit, the electromagnetic pulse circuit to generate the micro-current affecting the equalizing pulse of the human semicircular canal via the pulsed electromagnetic field after determining that the start condition is met, the method further includes: judging, by the processing circuit, whether a close condition of the electromagnetic pulse circuit is met according to an obtained current motion state of the user; and triggering, by the processing circuit, the electromagnetic pulse circuit to stop generating the micro-current after determining that the close condition is met.

In some embodiments, judging, by the processing circuit, whether the close condition is met according to the obtained current motion state of the user, includes: if the obtained current motion state includes the acceleration, judging, by the processing circuit, whether a current acceleration of the user is less than a second acceleration threshold set currently, and if so, determining that the close condition is met, otherwise determining that the close condition is not met; or if the obtained current motion state includes the moving distance in the horizontal direction per unit time, judging, by the processing circuit, whether a current moving distance in the horizontal direction per unit time of the user is less than a second horizontal distance threshold set currently, and if so, determining that the close condition is met, otherwise determining that the close condition is not met; or if the obtained current motion state includes the moving distance in the vertical direction per unit time, judging, by the processing circuit, whether a current moving distance in the vertical direction per unit time of the user is less than a second vertical distance threshold set currently, and if so, determining that the close condition is met, otherwise determining that the close condition is not met.

In some embodiments, the processing circuit determines the second acceleration threshold set currently by: taking an acceleration threshold corresponding to a current application scenario as the second acceleration threshold set currently according to a correspondence between application scenarios and acceleration thresholds; or the processing circuit determines the second horizontal distance threshold set currently by: taking a horizontal distance threshold corresponding to a current application scenario as the second horizontal distance threshold set currently according to a correspondence between application scenarios and horizontal distance thresholds; or the processing circuit determines the second vertical distance threshold set currently by: taking a vertical distance threshold corresponding to a current application scenario as the second vertical distance threshold set currently according to a correspondence between application scenarios and vertical distance thresholds.

In another aspect, the embodiments of the disclosure provide a system for preventing motion sickness, the system includes a processing circuit, a state sensing circuit and an electromagnetic pulse circuit, wherein the processing circuit is configured to: obtain a current motion state of a user via the state sensing circuit, judge whether a start condition of the electromagnetic pulse circuit is met according to the current motion state of the user, and trigger the electromagnetic pulse circuit to generate a micro-current affecting an equalizing pulse of a human semicircular canal via a pulsed electromagnetic field after determining that the start condition is met.

In some embodiments, the current motion state includes a part or all of: the acceleration, a moving distance in a horizontal direction per unit time, or a moving distance in a vertical direction per unit time.

In some embodiments, the processing circuit is configured to: if the current motion state includes the acceleration, judge whether a current acceleration of the user is greater than a first acceleration threshold set currently, and if so, determine that the start condition is met, otherwise determine that the start condition is not met; or if the current motion state includes the moving distance in the horizontal direction per unit time, judge whether a current moving distance in the horizontal direction per unit time of the user is greater than a first horizontal distance threshold set currently, and if so, determine that the start condition is met, otherwise determine that the start condition is not met; or if the current motion state includes the moving distance in the vertical direction per unit time, judge whether a current moving distance in the vertical direction per unit time of the user is greater than a first vertical distance threshold set currently, and if so, determine that the start condition is met, otherwise determine that the start condition is not met.

In some embodiments, the processing circuit is configured to determine the first acceleration threshold set currently by: taking an acceleration threshold corresponding to a current application scenario as the first acceleration threshold set currently according to a correspondence between application scenarios and acceleration thresholds; or the processing circuit is configured to determine the first horizontal distance threshold set currently by: taking a horizontal distance threshold corresponding to a current application scenario as the first horizontal distance threshold set currently according to a correspondence between application scenarios and horizontal distance thresholds; or the processing circuit is configured to determine the first vertical distance threshold set currently by: taking a vertical distance threshold corresponding to a current application scenario as the first vertical distance threshold set currently according to a correspondence between application scenarios and vertical distance thresholds.

In some embodiments, the processing circuit is further configured to: judge whether a close condition of the electromagnetic pulse circuit is met according to an obtained current motion state of the user; trigger the electromagnetic pulse circuit to stop generating the micro-current after determining that the close condition is met.

In some embodiments, the processing circuit is configured to: if the obtained current motion state includes the acceleration, judge whether a current acceleration of the user is less than a second acceleration threshold set currently, and if so, determine that the close condition is met, otherwise determine that the close condition is not met; or if the obtained current motion state includes the moving distance in the horizontal direction per unit time, judge whether a current moving distance in the horizontal direction per unit time of the user is less than a second horizontal distance threshold set currently, and if so, determine that the close condition is met, otherwise determine that the close condition is not met; or if the obtained current motion state includes the moving distance in the vertical direction per unit time, judge whether a current moving distance in the vertical direction per unit time of the user is less than a second vertical distance threshold set currently, and if so, determine that the close condition is met, otherwise determine that the close condition is not met.

In some embodiments, the processing circuit is configured to determine the second acceleration threshold set currently by: taking an acceleration threshold corresponding to a current application scenario as the second acceleration threshold set currently according to a correspondence between application scenarios and acceleration thresholds; or the processing circuit is configured to determine the second horizontal distance threshold set currently by: taking a horizontal distance threshold corresponding to a current application scenario as the second horizontal distance threshold set currently according to a correspondence between application scenarios and horizontal distance thresholds; or the processing circuit is configured to determine the second vertical distance threshold set currently by: taking a vertical distance threshold corresponding to a current application scenario as the second vertical distance threshold set currently according to a correspondence between application scenarios and vertical di stance thresholds.

In some embodiments, the processing circuit, the state sensing circuit and the electromagnetic pulse circuit are located in the same entity.

In some embodiments, the processing circuit is separately located in one entity, and the state sensing circuit and the electromagnetic pulse circuit are located in another entity.

In some embodiments, the state sensing circuit is separately located in one entity, and the processing circuit and the electromagnetic pulse circuit are located in another entity.

In some embodiments, each of the processing circuit, the state sensing circuit and the electromagnetic pulse circuit is located in one entity separately.

In some embodiments, the state sensing circuit is worn by the user, or is located on a transport in which the user sits.

In some embodiments, the state sensing circuit includes a Global Positioning System, or a gyroscope.

### BRIEF DESCRIPTION OF THE DRAWINGS

In order to make the technical solutions according to the embodiments of the disclosure more apparent, the drawings to which a description of the embodiments refers will be briefly introduced below, and apparently the drawings to be described below are merely illustrative of some of the embodiments of the disclosure, and those ordinarily skilled in the art can derive from these drawings other drawings without any inventive effort.

FIG. 1 is a first schematic flow diagram of a method for preventing motion sickness according to the embodiments of the disclosure;

FIG. 2 is a second schematic flow diagram of a method for preventing motion sickness according to the embodiments of the disclosure;

FIG. 3A is a schematic structural diagram of a system for preventing motion sickness according to the embodiments of the disclosure;

FIG. 3B is a schematic structural diagram of a system for preventing motion sickness where three circuits of the system are located in a same entity according to the embodiments of the disclosure;

FIG. 3C is a schematic structural diagram of a system for preventing motion sickness where a state sensing circuit and an electromagnetic pulse circuit of the system are located in one entity and a processing circuit of the system is separately located in another entity according to the embodiments of the disclosure;

FIG. 3D is a schematic structural diagram of a system for preventing motion sickness where a processing circuit and an electromagnetic pulse circuit of the system are located in one entity and a state sensing circuit of the system is separately located in another entity according to the embodiments of the disclosure;

FIG. 3E is a schematic structural diagram of a system for preventing motion sickness where each of a processing circuit, a state sensing circuit and an electromagnetic pulse circuit of the system is located in one entity separately according to the embodiments of the disclosure;

FIG. 4 is a schematic diagram of a position of a state sensing circuit located in a bus according to the embodiments of the disclosure;

FIG. 5 is a schematic structural diagram of an electromagnetic pulse circuit according to the embodiments of the disclosure;

FIG. 6 is a schematic diagram of a position of an electromagnetic pulse circuit located inside a headset according to the embodiments of the disclosure.

### DETAILED DESCRIPTION OF THE EMBODIMENTS

In the related art, treatments for the “motion sickness” mainly include life experience and medication.

First: the treatment of life experience and knowledge. People with motion sickness usually have symptoms such as dizziness, nausea, emesis, pale face, cold sweat and the like in the motion environment, and for the people with severe motion sickness, the symptoms such as arrhythmia, collapse, shock and the like even occur. Based on the universality of the “motion sickness”, people gathers some tips gradually, such as containing a ginger in the mouth, smelling the dry tree skin, applying the wind medicated oil or the like.

Second: the common medication treatment. Since there is no method of curing such disease thoroughly, the antihistamines and anticholinergic drugs can be selected.

The above-mentioned methods are obtained by the people in the daily practice. Where the first method is only applicable to the crowd with the slight “motion sickness”, and has no effect on the patients with the severer symptoms; while for the second method, firstly taking a transport on a business trip may be accidental and drugs may be forgotten to carry due to the hurry, and secondly taking the drugs for a long time may lead to a strong dependence on the drugs. Thus both the two methods are not good treatments for the “motion sickness”.

In conclusion, in the related art, the nausea, emesis and other symptoms caused by the “motion sickness” can only be prevented through the drugs with no other alternative way.

In order to make the objects, technical solutions, and advantages of the embodiments of the disclosure more apparent, the technical solutions according to the embodiments of the disclosure will be described below clearly and fully with reference to the drawings in the embodiments of the disclosure, and apparently the embodiments described below are only a part but not all of the embodiments of the disclosure. Based upon the embodiments here of the disclosure, all the other embodiments which can occur to those skilled in the art without any inventive effort shall fall into the scope of the disclosure.

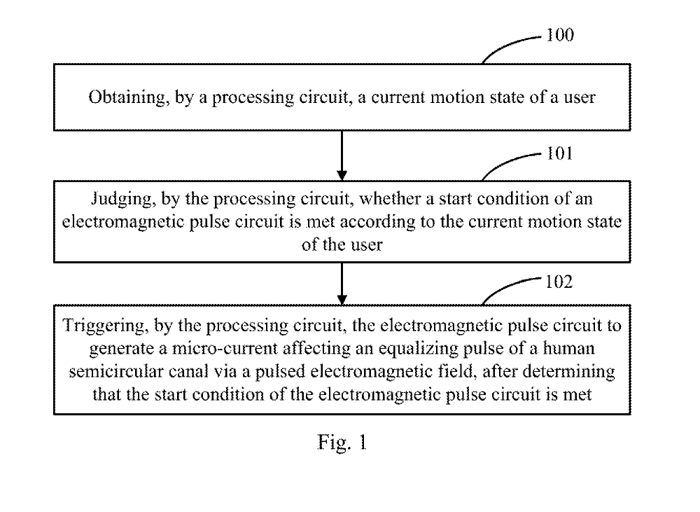

As illustrated in FIG. 1, the embodiments of the disclosure provide a method for preventing motion sickness, which includes following operations.

Operation 100: obtaining, by a processing circuit, a current motion state of a user.

Operation 101: judging, by the processing circuit, whether a start condition of an electromagnetic pulse circuit is met according to the current motion state of the user.

Operation 102: triggering, by the processing circuit, the electromagnetic pulse circuit to generate a micro-current affecting an equalizing pulse of a human semicircular canal via a pulsed electromagnetic field, after determining that the start condition of the electromagnetic pulse circuit is met.

In the embodiments of the disclosure, the processing circuit obtains the current motion state of the user, and judges whether the start condition of the electromagnetic pulse circuit is met according to the current motion state of the user. If the start condition of the electromagnetic pulse circuit is met, the electromagnetic pulse circuit generates the micro-current affecting the equalizing pulse of the human semicircular canal via the pulsed electromagnetic field. In the embodiments of the disclosure, the micro-current affecting the equalizing pulse of the human semicircular canal is generated after the motion state meets the start condition, to thereby prevent the symptoms such as nausea, emesis or the like caused by the “motion sickness” and effectively solve the problem in the related art that the way of preventing the motion sickness is unitary.

It shall be noted that, the micro-current according to the embodiments of the disclosure is a current affecting the equalizing pulse of the human semicircular canal, where the current lies in a normal current range which can be withstood by the human body, for example, the current generally ranges from 0 to 500 μA.

Since it is easy for a user to cause motion sickness when the equipment in which the user sits is in a state of acceleration, deceleration, shaking or another similar state, it can be judged whether the user is in the state of acceleration, deceleration, shaking or another similar state or not according to the current motion state of the user, and if so, the starting of the micro-current can be triggered to thereby achieve the effect of preventing the motion sickness.

In some embodiments, the current motion state of the user according to the embodiments of the disclosure includes but not limited to a part or all of: acceleration, a moving distance in a horizontal direction per unit time, or a moving distance in a vertical direction per unit time.

In some embodiments, the acceleration mainly refers to the acceleration in the horizontal direction.

In some embodiments, the ways of obtaining the acceleration include but not limited to following approaches.

The first approach: a speed of a starting point is denoted as V₀, a speed of an ending point is denoted as Vₜ, and a time required from the starting point to the ending point is t. Then, the acceleration a=(Vₜ−V₀)/t.

The second approach: the speed of the starting point is denoted as V₀, the speed of the ending point is denoted as Vₜ, and a path from the starting point to the ending point is s. Then, the acceleration a=(Vₜ²−V₀²)/2s.

In some embodiments, the speeds of the starting point and the ending point of the user are obtained by a speed sensor; and the time is obtained by a timer.

In some embodiments, the moving distance in the horizontal direction per unit time is also the path said generally, i.e., the whole moving distance of the user from the starting point to the ending point.

For example, the distance of the user from the point a to the point b is 20 meters, while the actual moving distance of the user reaches 25 meters, that is, the path is 25 meters, so the moving distance in the horizontal direction per unit time is denoted as 25 meters.

In some embodiments, the moving distance in the vertical direction per unit time is also the path said generally, i.e., the whole moving distance of the user from the starting point to the ending point.

For example, when the user takes a ship, the trail of the ship in the vertical direction may change due to the flood or ebb tide of the sea or other factors. At this time, since the ship moves at a constant speed on one hand and is affected by the sea on the other hand, the ship may generate a displacement along a forward diagonal direction. At this time, the moving distance in the vertical direction per unit time is still the path on which the ship moves actually.

For example, the ship moves 1000 meters actually, the displacement in the vertical direction is 500 meters, and then the moving distance in the vertical direction per unit time is denoted as 1000 meters.

The motion states described above substantially include all the scenarios where the “motion sickness” is easily generated. Generally walking on the horizontal pavement only needs to use such motion state as the acceleration. The other scenarios such as ship, airplane or the like need to use two motion states of the moving distance in the horizontal direction per unit time and the moving distance in the vertical direction per unit time. In a practical implementation, of course, different motion states can also be selected according to different application scenarios: the way of using only one motion state, or the way of using two combined motion states, or the way of using three co-existing motion states.

In some embodiments, after obtaining the current motion state of the user, judging, by the processing circuit, whether the start condition is met according to the current motion state of the user, includes: if the current motion state includes the acceleration, judging, by the processing circuit, whether a current acceleration of the user is greater than a first acceleration threshold set currently, and if so, determining that the start condition is met, otherwise determining that the start condition is not met; or, if the current motion state includes the moving distance in the horizontal direction per unit time, judging, by the processing circuit, whether a current moving distance in the horizontal direction per unit time of the user is greater than a first horizontal distance threshold set currently, and if so, determining that the start condition is met, otherwise determining that the start condition is not met; or, if the current motion state includes the moving distance in the vertical direction per unit time, judging, by the processing circuit, whether a current moving distance in the vertical direction per unit time of the user is greater than a first vertical distance threshold set currently, and if so, determining that the start condition is met, otherwise determining that the start condition is not met.

The start condition described herein is: whether to trigger the electromagnetic pulse circuit to work.

That is, if the start condition is met, the processing circuit triggers the electromagnetic pulse circuit to generate the micro-current affecting the equalizing pulse of the human semicircular canal via the pulsed electromagnetic field; and conversely, the processing circuit does not trigger the electromagnetic pulse circuit to generate the micro-current affecting the equalizing pulse of the human semicircular canal via the pulsed electromagnetic field, or the processing circuit triggers the electromagnetic pulse circuit not to generate the micro-current affecting the equalizing pulse of the human semicircular canal via the pulsed electromagnetic field.

This method can obtains the current motion state of the user in real time and detects in real time whether the obtained current motion state of the user needs to trigger the electromagnetic pulse circuit to work according to the obtained motion state of the user, to prevent the occurrence of the “motion sickness” in real time.

In some embodiments, there are three ways to determine the first threshold set currently according to different motion states corresponding to the current application scenario, which are as follows.

First, if the obtained motion state of the user is the acceleration, the processing circuit determines the first acceleration threshold set currently by: taking an acceleration threshold corresponding to a current application scenario as the first acceleration threshold set currently according to a correspondence between application scenarios and acceleration thresholds.

Second, if the obtained motion state of the user is the moving distance in the horizontal direction per unit time, the processing circuit determines the first horizontal distance threshold set currently by: taking a horizontal distance threshold corresponding to a current application scenario as the first horizontal distance threshold set currently according to a correspondence between application scenarios and horizontal distance thresholds.

Third, if the obtained motion state of the user is the moving distance in the vertical direction per unit time, the processing circuit determines the first vertical distance threshold set currently by: taking a vertical distance threshold corresponding to a current application scenario as the first vertical distance threshold set currently according to a correspondence between application scenarios and vertical distance thresholds.

In some embodiments, there are following ways to determine the current application scenario.

First, the user selects the application scenario, and the user selects the range of the first threshold autonomously according to the transport in which he sits.

Second, the application scenario of the user is judged according to the current speed of the user. For example, if the speed of the user is always above 500 for a period of time, the application scenario is selected as the airplane; if the speed of the user is always between 200 and 300 for a period of time, the application scenario is selected as the train; if the speed of the user is always between 60 and 130 for a period of time, the application scenario is selected as the car; if the speed of the user is always between 40 and 80 for a period of time, the application scenario is selected as the ship.

The processing circuit obtains the corresponding first acceleration threshold, the corresponding first horizontal distance threshold and the corresponding first vertical distance threshold respectively by the three ways described above, to distinguish the different first acceleration thresholds, the different first horizontal distance thresholds and the different first vertical distance thresholds set when the user is in different application scenarios; and it facilitates the processing circuit to judge whether the electromagnetic pulse circuit needs to work according to the different first acceleration thresholds, the different first horizontal distance thresholds and the different first vertical distance thresholds in the different application scenarios.

That is to say, the first thresholds set when the user is in different transports are different.

For example, if the user is in a train, the first threshold can be set to 100; if the user is in an airplane, the first threshold can be set to 800.

The different users can also adjust the first threshold according to the personal requirements, for example, the user A needs to set the first threshold to 500 to avoid the occurrence of the “motion sickness” when he is in the airplane, while the user B may need to set the first threshold to 1000 to avoid the occurrence of the “motion sickness”.

Furthermore, the setting of the first threshold is not simplex.

For example, when the airplane is in the motion state of spiraling up, the way of combining the moving distance in the horizontal direction per unit time with the moving distance in the vertical direction per unit time can be adopted; where the first horizontal distance threshold is set to 500, and the first vertical distance threshold is set to 1000. In this case, the processing circuit triggers the electromagnetic pulse circuit to work so long as one of the moving distance in the horizontal direction per unit time and the moving distance in the vertical direction per unit time is greater than the first threshold. Of course, it can also be configured so that the processing circuit triggers the electromagnetic pulse circuit to work only when both the moving distance in the horizontal direction per unit time and the moving distance in the vertical direction per unit time are greater than the first threshold.

For the several approaches described above, the user can adjust the first threshold properly according to the personal requirements in the practice.

In some embodiments, after the processing circuit compares with the set first thresholds of different motion states to obtain the conclusion that the start condition is met, and triggers the electromagnetic pulse circuit to generate the micro-current, which affects the equalizing pulse of the human semicircular canal, via the pulsed electromagnetic field, the method further includes: judging, by the processing circuit, whether a close condition of the electromagnetic pulse circuit is met according to an obtained current motion state of the user; triggering, by the processing circuit, the electromagnetic pulse circuit to stop generating the micro-current after determining that the close condition of the electromagnetic pulse circuit is met.

That is to say, after the processing circuit triggers the electromagnetic pulse circuit to generate the micro-current affecting the equalizing pulse of the human semicircular canal via the pulsed electromagnetic field, the processing circuit further determines whether there is a need to close the operation of the electromagnetic pulse circuit according to the obtained motion state of the user. If the closing is not in time, it may cause some other diseases. The state of the user can be detected in real time only based on the detected current motion state of the user. If the user has any discomfort symptom under the current motion state, a feedback can be given to the processing circuit in real time, so that the processing circuit triggers the electromagnetic pulse circuit to work in real time if needed, to thereby prevent the occurrence of the “motion sickness” effectively.

In some embodiments, the embodiments of the disclosure provide a way of turning off the electromagnetic pulse circuit automatically besides turning off the electromagnetic pulse circuit manually.

The way of turning off the electromagnetic pulse circuit automatically can avoid the obsession caused by the fact that the user forgets to turn off the electromagnetic pulse circuit due to the hurry or haste in time.

In the embodiments of the disclosure, after the processing circuit compares with the set first thresholds of different motion states to obtain the conclusion that the start condition is met, and triggers the electromagnetic pulse circuit to generate the micro-current, which affects the equalizing pulse of the human semicircular canal, via the pulsed electromagnetic field, there is also a need to judge whether the close condition of the electromagnetic pulse circuit is met according to the obtained current motion state of the user. Where three ways of judging whether the close condition is met are provided as follows.

First, if the obtained current motion state includes the acceleration, judging, by the processing circuit, whether the current acceleration of the user is less than a second acceleration threshold set currently, and if so, determining that the close condition is met, otherwise determining that the close condition is not met.

Second, if the obtained current motion state includes the moving distance in the horizontal direction per unit time, judging, by the processing circuit, whether the current moving distance in the horizontal direction per unit time of the user is less than a second horizontal distance threshold set currently, and if so, determining that the close condition is met, otherwise determining that the close condition is not met.

Third, if the obtained current motion state includes the moving distance in the vertical direction per unit time, judging, by the processing circuit, whether the current moving distance in the vertical direction per unit time of the user is less than a second vertical distance threshold set currently, and if so, determining that the close condition is met, otherwise determining that the close condition is not met.

The second acceleration threshold, the second horizontal distance threshold and the second vertical distance threshold corresponding to different motion states are set according to different application scenarios, to distinguish that the user is in the different application scenarios, and facilitate the processing circuit to judge whether the electromagnetic pulse circuit needs to stop working according to the different second acceleration thresholds, the different second horizontal distance thresholds and the different second vertical distance thresholds in the different application scenarios.

For example, the second thresholds are different when the user is in the bus, airplane or ship, and such design is more reasonable.

In some embodiments, there are three ways to determine the second threshold set currently according to different motion states corresponding to the current application scenario, which are as follows.

First, if the obtained current motion state of the user is the acceleration, the processing circuit determines the second acceleration threshold set currently by: taking an acceleration threshold corresponding to a current application scenario as the second acceleration threshold set currently according to a correspondence between application scenarios and acceleration thresholds.

Second, if the obtained current motion state of the user is the moving distance in the horizontal direction per unit time, the processing circuit determines the second horizontal distance threshold set currently by: taking a horizontal distance threshold corresponding to a current application scenario as the second horizontal distance threshold set currently according to a correspondence between application scenarios and horizontal distance thresholds.

Third, if the obtained current motion state of the user is the moving distance in the vertical direction per unit time, the processing circuit determines the second vertical distance threshold set currently by: taking a vertical distance threshold corresponding to a current application scenario as the second vertical distance threshold set currently according to a correspondence between application scenarios and vertical distance thresholds.

The processing circuit obtains the corresponding second acceleration threshold, the corresponding second horizontal distance threshold and the corresponding second vertical distance threshold respectively by the three ways described above, to distinguish the different second acceleration thresholds, the different second horizontal distance thresholds and the different second vertical distance thresholds set when the user is in different application scenarios. It facilitates the processing circuit to judge whether the electromagnetic pulse circuit needs to stop working according to the different second acceleration thresholds, the different second horizontal distance thresholds and the different second vertical distance thresholds in the different application scenarios.

Here, the setting of the second threshold corresponds to the setting of the first threshold described above.

In conclusion, there is a need for the setting of the thresholds to distinguish different application scenarios and different users. The “motion sickness” may be slightly different according to different ages, sexes, physiques and the like, and there is a need to distinguish them according to different scenarios and users.

The complete flow of a method for preventing motion sickness of the embodiments of the disclosure will be introduced below by taking as an example that the motion state is the acceleration.

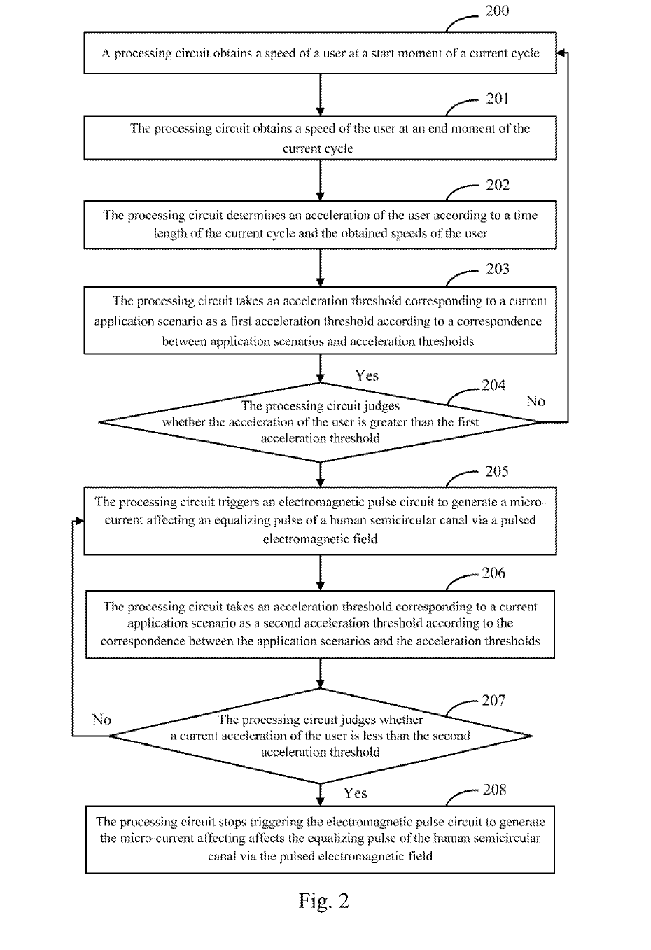

As illustrated in FIG. 2, the method for preventing the motion sickness according to the embodiments of the disclosure includes the following operations.

Operation 200: a processing circuit obtains a speed of a user at a start moment of a current cycle.

Operation 201: the processing circuit obtains a speed of the user at an end moment of the current cycle.

Operation 202: the processing circuit determines an acceleration of the user according to a time length of the current cycle and the obtained speeds of the user.

Operation 203: the processing circuit takes an acceleration threshold corresponding to a current application scenario as a first acceleration threshold according to a correspondence between application scenarios and acceleration thresholds.

Operation 204: the processing circuit judges whether the acceleration of the user is greater than the first acceleration threshold; if so, the operation 205 is performed; otherwise the operation 200 is performed.

Operation 205: the processing circuit triggers an electromagnetic pulse circuit to generate a micro-current affecting an equalizing pulse of a human semicircular canal via a pulsed electromagnetic field.

Operation 206: the processing circuit takes an acceleration threshold corresponding to a current application scenario as a second acceleration threshold according to the correspondence between the application scenarios and the acceleration thresholds.

Operation 207: the processing circuit judges whether a current acceleration of the user is less than the second acceleration threshold; if so, the operation 208 is performed, otherwise the operation 205 is performed.

Operation 208: the processing circuit stops triggering the electromagnetic pulse circuit to generate the micro-current affecting affects the equalizing pulse of the human semicircular canal via the pulsed electromagnetic field.

It shall be noted that, other motion states are similar to such motion state as the acceleration, thus repeated description thereof will be omitted here.

In some embodiments, one or more of the first acceleration threshold, the first horizontal distance threshold and the first vertical distance threshold can be selected as the first threshold according to different application scenarios; and similarly, one or more of the second acceleration threshold, the second horizontal distance threshold and the second vertical distance threshold can be selected as the second threshold according to different application scenarios.

Based upon the same inventive concept, the embodiments of the disclosure further provide a system for preventing motion sickness. Since the principle addressing the problem of the system is similar to the principle addressing the problem of the method for preventing motion sickness above according to the embodiments of the disclosure, the implementations of the system can refer to the implementations of the method above, thus repeated description thereof will be omitted here.

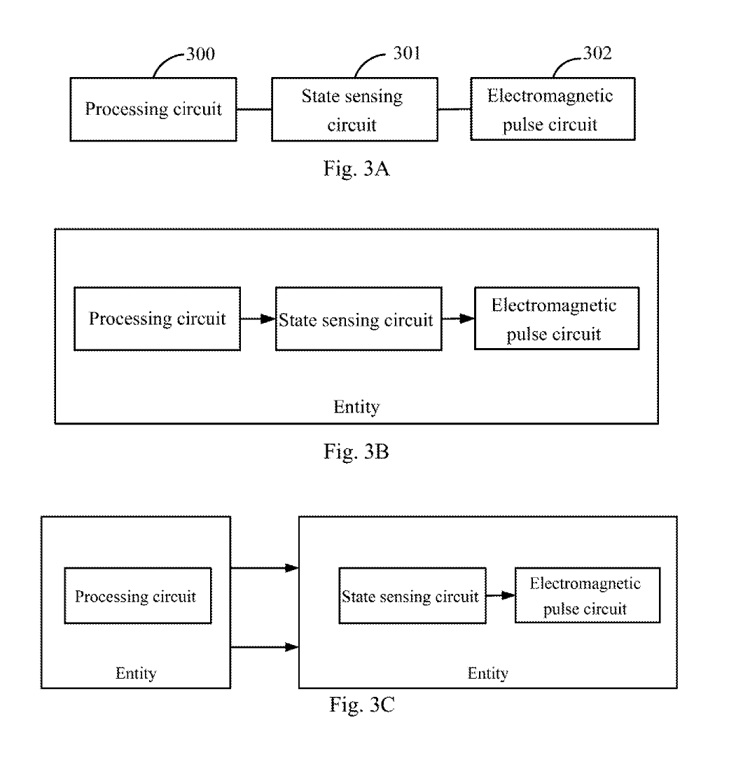

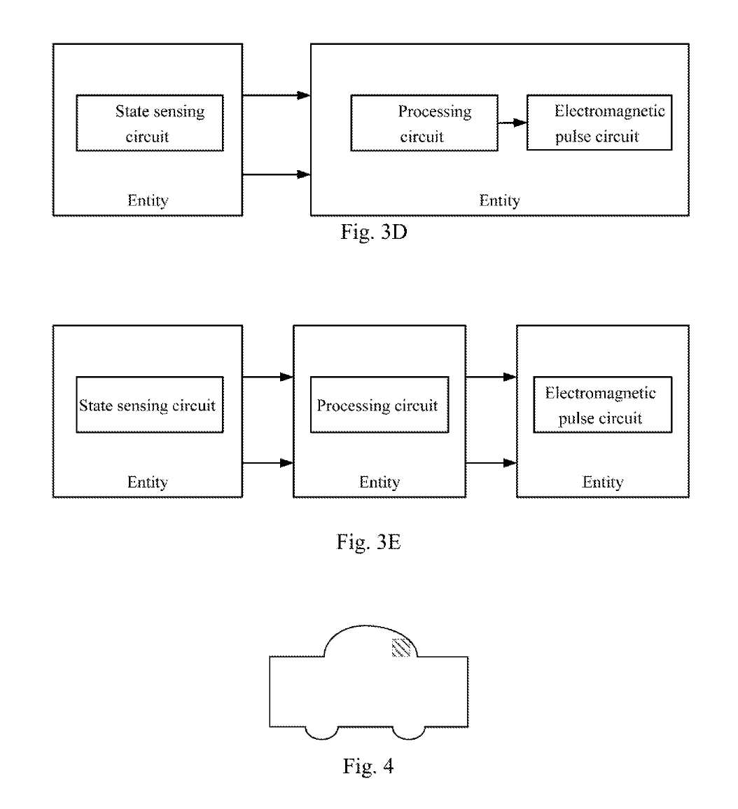

As illustrated in FIG. 3A, the embodiments of the disclosure provide a system for preventing motion sickness, which includes: a processing circuit 300, a state sensing circuit 301 and an electromagnetic pulse circuit 302.

The processing circuit 300 is configured to: obtain a current motion state of a user via the state sensing circuit, judge whether a start condition of the electromagnetic pulse circuit is met according to the current motion state of the user, and trigger the electromagnetic pulse circuit to generate a micro-current affecting an equalizing pulse of a human semicircular canal via a pulsed electromagnetic field after determining that the start condition is met.

Here, the state sensing circuit is a GPS (Global Positioning System), a gyroscope or another device. The GPS can obtain a position where the user is located. Further, an initial speed and a termination speed of the user are obtained by a speed sensor, to thereby obtain the acceleration; and the gyroscope herein is mainly a sensing gyroscope, which can obtain the horizontal, vertical, pitch, azimuth and other parameters.

In some embodiments, in order to obtain such motion state as the acceleration, the GPS is adopted; and alternatively, in order to obtain the moving distance in the horizontal direction per unit time and/or the moving distance in the vertical direction per unit time, such device as the gyroscope is adopted.

In some embodiments, the processing circuit 300, the state sensing circuit 301 and the electromagnetic pulse circuit 302 are located in the same entity, as illustrated in FIG. 3B.

Such structure is simple and easy to use.

In some embodiments, the processing circuit 300 is separately located in one entity, and the state sensing circuit 301 and the electromagnetic pulse circuit 302 are located in another entity, as illustrated in FIG. 3C.

In this solution, the processing circuit can be located in a mobile phone or another mobile device. Further, the processing circuit can be installed in the form of software, the on-off of the device can be realized via a mobile client, and the threshold parameters related to the operations of the electromagnetic pulse circuit can be adjusted via the mobile client to make it applicable to varying degrees of motion sickness patients.

In some embodiments, the state sensing circuit 301 is separately located in one entity, and the processing circuit 300 and the electromagnetic pulse circuit 302 are located in another entity, as illustrated in FIG. 3D.

In the use procedure of such encapsulation approach, the state sensing circuit 301 can be fixed onto the transport, which facilitates the more accurate tracking of the motion parameters and simultaneously reduces the obsession of the too large whole structure because the processing circuit 300, the state sensing circuit 301 and the electromagnetic pulse circuit 302 are located in the same entity. Where the communications among the state sensing circuit 301, the processing circuit 300 and the electromagnetic pulse circuit 302 can be performed in form of wired or wireless signals.

In some embodiments, each of the processing circuit 300, the state sensing circuit 301 and the electromagnetic pulse circuit 302 is located in one entity separately, as illustrated in FIG. 3E.

Such structure is easy for the user to distinguish each of the circuits and not confuse.

In some embodiments, the state sensing circuit can be worn by the user, or can be located on the transport in which the user sits; if the state sensing circuit is located on the transport, it should be located in a certain position which is relatively stable. For example, if the transport is an airplane, it is best for the state sensing circuit to be located in a certain position inside the airplane, but not on the airfoil relatively at the outside. If the transport is a bus, the state sensing circuit only needs to be located inside the bus, for example, the dotted frame as illustrated in FIG. 4 represents the state sensing circuit, which is located at the front window of the bus. It shall be noted that, FIG. 4 is only a schematic diagram of the position of the state sensing circuit, and in practice, it can also be located at the back window or on the seat of the bus so long as the position is relatively fixed.

If the transport is an airplane or a ship, the state sensing circuit can be located at a certain position inside the airplane or ship. With respect to the airplane and ship, the sensors like the nerve of the airplane and ship, spread all over the airplane and ship, transform each operating state parameter of the airplane and ship into an electrical signal, and then transmit the signal, to escort the security of the airplane and ship.

This facilitates the state sensing circuit to better sense the current motion state of the user, so that the processing circuit judges whether the electromagnetic pulse circuit needs to be triggered to generate the micro-current affecting the equalizing pulse of the human semicircular canal via the pulsed electromagnetic field according to the obtained current motion state of the user.

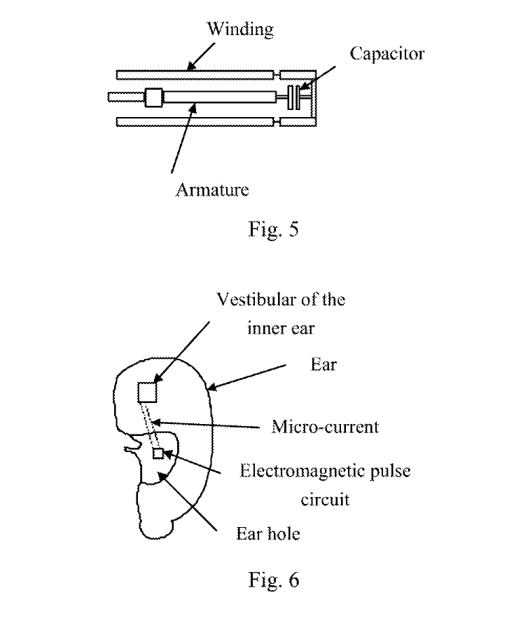

In some embodiments, the schematic structural diagram of the electromagnetic pulse circuit is as illustrated in FIG. 5, where the electromagnetic pulse circuit includes a winding, an armature, and a capacitor, etc.

In some embodiments, the above-mentioned system can be encapsulated into a same electronic device, i.e., encapsulated into a headphone, so the electromagnetic pulse circuit should be located at the position inside the headphone and close to the human semicircular canal, as illustrated in FIG. 6. In this figure, the position of the electromagnetic pulse circuit is close to the human semicircular canal, when the state sensing circuit obtains the current motion state of the user, the processing circuit judges whether the start condition of the electromagnetic pulse circuit is met according to the obtained current motion state of the user, and will trigger the electromagnetic pulse circuit to generate the micro-current affecting the equalizing pulse of the human semicircular canal via the pulsed electromagnetic field after determining that the start condition is met.

The purpose of doing this is to transmit the micro-current to the human semicircular canal in real time, and better prevent the occurrence of the “motion sickness”.

In some embodiments, the current motion state includes a part or all of: the acceleration, a moving distance in a horizontal direction per unit time, or a moving distance in a vertical direction per unit time.

In some embodiments, the processing circuit 300 is configured to: if the current motion state includes the acceleration, judge whether a current acceleration of the user is greater than a first acceleration threshold set currently, and if so, determine that the start condition is met, otherwise determine that the start condition is not met; or if the current motion state includes the moving distance in the horizontal direction per unit time, judge whether a current moving distance in the horizontal direction per unit time of the user is greater than a first horizontal distance threshold set currently, and if so, determine that the start condition is met, otherwise determine that the start condition is not met; or if the current motion state includes the moving distance in the vertical direction per unit time, judge whether a current moving distance in the vertical direction per unit time of the user is greater than a first vertical distance threshold set currently, and if so, determine that the start condition is met, otherwise determine that the start condition is not met.

In some embodiments, the processing circuit 300 determines the first acceleration threshold set currently by: taking an acceleration threshold corresponding to a current application scenario as the first acceleration threshold set currently according to a correspondence between application scenarios and acceleration thresholds; or the processing circuit 300 determines the first horizontal distance threshold set currently by: taking a horizontal distance threshold corresponding to a current application scenario as the first horizontal distance threshold set currently according to a correspondence between application scenarios and horizontal distance thresholds; or the processing circuit 300 determines the first vertical distance threshold set currently by: taking a vertical distance threshold corresponding to a current application scenario as the first vertical distance threshold set currently according to a correspondence between application scenarios and vertical distance thresholds.

In some embodiments, the processing circuit 300 is further configured to: judge whether a close condition of the electromagnetic pulse circuit is met according to an obtained current motion state of the user; trigger the electromagnetic pulse circuit to stop generating the micro-current after determining that the close condition is met.

In some embodiments, the processing circuit 300 is configured to: if the obtained current motion state includes the acceleration, judge whether a current acceleration of the user is less than a second acceleration threshold set currently, and if so, determine that the close condition is met, otherwise determine that the close condition is not met; or if the obtained current motion state includes the moving distance in the horizontal direction per unit time, judge whether a current moving distance in the horizontal direction per unit time of the user is less than a second horizontal distance threshold set currently, and if so, determine that the close condition is met, otherwise determine that the close condition is not met; or if the obtained current motion state includes the moving distance in the vertical direction per unit time, judge whether a current moving distance in the vertical direction per unit time of the user is less than a second vertical distance threshold set currently, and if so, determine that the close condition is met, otherwise determine that the close condition is not met.

In some embodiments, the processing circuit 300 determines the second acceleration threshold set currently by: taking an acceleration threshold corresponding to a current application scenario as the second acceleration threshold set currently according to a correspondence between application scenarios and acceleration thresholds; or the processing circuit 300 determines the second horizontal distance threshold set currently by: taking a horizontal distance threshold corresponding to a current application scenario as the second horizontal distance threshold set currently according to a correspondence between application scenarios and horizontal distance thresholds; or the processing circuit 300 determines the second vertical distance threshold set currently by: taking a vertical distance threshold corresponding to a current application scenario as the second vertical distance threshold set currently according to a correspondence between application scenarios and vertical distance thresholds.

The disclosure has been described above by reference to the block diagrams and/or flow charts showing the methods, devices (systems) and/or computer program products according to the embodiments of the disclosure. It should be understood that one block illustrated in the block diagrams and/or flow charts and a combination of the blocks illustrated in the block diagrams and/or flow charts can be implemented by the computer program instructions. These computer program instructions can be provided to a general-purpose computer, a processor of a dedicated computer and/or another programmable data processing unit to generate a machine, so that the instructions executed by the computer processor and/or another programmable data processing unit create the methods for implementing the functions and/or actions specified in the blocks of the block diagrams and/or flow charts.

Accordingly, the disclosure can also be implemented by the hardware and/or software (including the firmware, resident software, microcode and the like). Further, the disclosure can adopt the form of computer program products on the computer usable or computer readable storage medium, which has the computer usable or computer readable program codes implemented in the medium, to be used by the instruction execution system or used in combination with the instruction execution system. In the context of the disclosure, the computer usable or computer readable storage medium can be any medium, which can contain, store, communicate with, transmit or transfer the programs, to be used by the instruction execution system, apparatus or device, or used in combination with the instruction execution system, apparatus or device.

Evidently those skilled in the art can make various modifications and variations to the disclosure without departing from the spirit and scope of the disclosure. Thus the disclosure is also intended to encompass these modifications and variations therein as long as these modifications and variations come into the scope of the claims of the disclosure and their equivalents.

1. A method for preventing motion sickness, comprising:
obtaining, by a processing circuit, a current motion state of a user;
judging, by the processing circuit, whether a start condition of an electromagnetic pulse circuit is met according to the current motion state of the user; and
triggering, by the processing circuit, the electromagnetic pulse circuit to generate a micro-current affecting an equalizing pulse of a human semicircular canal via a pulsed electromagnetic field, after determining that the start condition is met.

2. The method according to claim 1, wherein the current motion state comprises a part or all of:
an acceleration, a moving distance in a horizontal direction per unit time, or a moving distance in a vertical direction per unit time.

3. The method according to claim 2, wherein judging, by the processing circuit, whether the start condition is met according to the current motion state of the user, comprises:
if the current motion state comprises the acceleration, judging, by the processing circuit, whether a current acceleration of the user is greater than a first acceleration threshold set currently, and if so, determining that the start condition is met, otherwise determining that the start condition is not met; or
if the current motion state comprises the moving distance in the horizontal direction per unit time, judging, by the processing circuit, whether a current moving distance in the horizontal direction per unit time of the user is greater than a first horizontal distance threshold set currently, and if so, determining that the start condition is met, otherwise determining that the start condition is not met; or
if the current motion state comprises the moving distance in the vertical direction per unit time, judging, by the processing circuit, whether a current moving distance in the vertical direction per unit time of the user is greater than a first vertical distance threshold set currently, and if so, determining that the start condition is met, otherwise determining that the start condition is not met.

4. The method according to claim 3, wherein the processing circuit determines the first acceleration threshold set currently by: taking an acceleration threshold corresponding to a current application scenario as the first acceleration threshold set currently according to a correspondence between application scenarios and acceleration thresholds; or
the processing circuit determines the first horizontal distance threshold set currently by: taking a horizontal distance threshold corresponding to a current application scenario as the first horizontal distance threshold set currently according to a correspondence between application scenarios and horizontal distance thresholds; or
the processing circuit determines the first vertical distance threshold set currently by: taking a vertical distance threshold corresponding to a current application scenario as the first vertical distance threshold set currently according to a correspondence between application scenarios and vertical distance thresholds.

5. The method according to claim 2, wherein after triggering, by the processing circuit, the electromagnetic pulse circuit to generate the micro-current affecting the equalizing pulse of the human semicircular canal via the pulsed electromagnetic field after determining that the start condition is met, the method further comprises:
judging, by the processing circuit, whether a close condition of the electromagnetic pulse circuit is met according to an obtained current motion state of the user; and
triggering, by the processing circuit, the electromagnetic pulse circuit to stop generating the micro-current after determining that the close condition is met.

6. The method according to claim 5, wherein judging, by the processing circuit, whether the close condition is met according to the obtained current motion state of the user, comprises:
if the obtained current motion state comprises the acceleration, judging, by the processing circuit, whether a current acceleration of the user is less than a second acceleration threshold set currently, and if so, determining that the close condition is met, otherwise determining that the close condition is not met; or
if the obtained current motion state comprises the moving distance in the horizontal direction per unit time, judging, by the processing circuit, whether a current moving distance in the horizontal direction per unit time of the user is less than a second horizontal distance threshold set currently, and if so, determining that the close condition is met, otherwise determining that the close condition is not met; or
if the obtained current motion state comprises the moving distance in the vertical direction per unit time, judging, by the processing circuit, whether a current moving distance in the vertical direction per unit time of the user is less than a second vertical distance threshold set currently, and if so, determining that the close condition is met, otherwise determining that the close condition is not met.

7. The method according to claim 6, wherein the processing circuit determines the second acceleration threshold set currently by: taking an acceleration threshold corresponding to a current application scenario as the second acceleration threshold set currently according to a correspondence between application scenarios and acceleration thresholds; or
the processing circuit determines the second horizontal distance threshold set currently by: taking a horizontal distance threshold corresponding to a current application scenario as the second horizontal distance threshold set currently according to a correspondence between application scenarios and horizontal distance thresholds; or
the processing circuit determines the second vertical distance threshold set currently by: taking a vertical distance threshold corresponding to a current application scenario as the second vertical distance threshold set currently according to a correspondence between application scenarios and vertical distance thresholds.

8. A system for preventing motion sickness, comprising a processing circuit, a state sensing circuit and an electromagnetic pulse circuit, wherein:
the processing circuit is configured to: obtain a current motion state of a user via the state sensing circuit, judge whether a start condition of the electromagnetic pulse circuit is met according to the current motion state of the user, and trigger the electromagnetic pulse circuit to generate a micro-current affecting an equalizing pulse of a human semicircular canal via a pulsed electromagnetic field after determining that the start condition is met.

9. The system according to claim 8, wherein the current motion state comprises a part or all of:
an acceleration, a moving distance in a horizontal direction per unit time, or a moving distance in a vertical direction per unit time.

10. The system according to claim 9, wherein the processing circuit is configured to:
if the current motion state comprises the acceleration, judge whether a current acceleration of the user is greater than a first acceleration threshold set currently, and if so, determine that the start condition is met, otherwise determine that the start condition is not met; or
if the current motion state comprises the moving distance in the horizontal direction per unit time, judge whether a current moving distance in the horizontal direction per unit time of the user is greater than a first horizontal distance threshold set currently, and if so, determine that the start condition is met, otherwise determine that the start condition is not met; or
if the current motion state comprises the moving distance in the vertical direction per unit time, judge whether a current moving distance in the vertical direction per unit time of the user is greater than a first vertical distance threshold set currently, and if so, determine that the start condition is met, otherwise determine that the start condition is not met.

11. The system according to claim 10, wherein the processing circuit is configured to determine the first acceleration threshold set currently by: taking an acceleration threshold corresponding to a current application scenario as the first acceleration threshold set currently according to a correspondence between application scenarios and acceleration thresholds; or
the processing circuit is configured to determine the first horizontal distance threshold set currently by: taking a horizontal distance threshold corresponding to a current application scenario as the first horizontal distance threshold set currently according to a correspondence between application scenarios and horizontal distance thresholds; or
the processing circuit is configured to determine the first vertical distance threshold set currently by: taking a vertical distance threshold corresponding to a current application scenario as the first vertical distance threshold set currently according to a correspondence between application scenarios and vertical distance thresholds.

12. The system according to claim 9, wherein the processing circuit is further configured to:
judge whether a close condition of the electromagnetic pulse circuit is met according to an obtained current motion state of the user; and
trigger the electromagnetic pulse circuit to stop generating the micro-current after determining that the close condition is met.

13. The system according to claim 12, wherein the processing circuit is configured to:
if the obtained current motion state comprises the acceleration, judge whether a current acceleration of the user is less than a second acceleration threshold set currently, and if so, determine that the close condition is met, otherwise determine that the close condition is not met; or
if the obtained current motion state comprises the moving distance in the horizontal direction per unit time, judge whether a current moving distance in the horizontal direction per unit time of the user is less than a second horizontal distance threshold set currently, and if so, determine that the close condition is met, otherwise determine that the close condition is not met; or
if the obtained current motion state comprises the moving distance in the vertical direction per unit time, judge whether a current moving distance in the vertical direction per unit time of the user is less than a second vertical distance threshold set currently, and if so, determine that the close condition is met, otherwise determine that the close condition is not met.

14. The system according to claim 13, wherein the processing circuit is configured to determine the second acceleration threshold set currently by: taking an acceleration threshold corresponding to a current application scenario as the second acceleration threshold set currently according to a correspondence between application scenarios and acceleration thresholds; or
the processing circuit is configured to determine the second horizontal distance threshold set currently by: taking a horizontal distance threshold corresponding to a current application scenario as the second horizontal distance threshold set currently according to a correspondence between application scenarios and horizontal distance thresholds; or
the processing circuit is configured to determine the second vertical distance threshold set currently by: taking a vertical distance threshold corresponding to a current application scenario as the second vertical distance threshold set currently according to a correspondence between application scenarios and vertical distance thresholds.

15. The system according to claim 8, wherein the processing circuit, the state sensing circuit and the electromagnetic pulse circuit are located in a same entity.

16. The system according to claim 8, wherein the processing circuit is separately located in one entity, and the state sensing circuit and the electromagnetic pulse circuit are located in another entity.

17. The system according to claim 8, wherein the state sensing circuit is separately located in one entity, and the processing circuit and the electromagnetic pulse circuit are located in another entity.

18. The system according to claim 8, wherein each of the processing circuit, the state sensing circuit and the electromagnetic pulse circuit is located in one entity separately.

19. The system according to claim 8, wherein the state sensing circuit is worn by the user, or is located on a transport in which the user sits.

20. The system according to claim 8, wherein the state sensing circuit comprises a Global Positioning System, or a gyroscope.
